(19) United States
(12) Patent Application Publication
(10) Pub. No.: US 2010/0105085 A1
(43) Pub. Date: Apr. 29, 2010

(54) METHOD OF DIAGNOSING AND STRATIFYING ANTI-PHOSPHOLIPID SYNDROME

(76) Inventors: Nir Dotan, Shoham (IL); Avinoam Dukler, Moddin (IL)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/310,183

(22) PCT Filed: Aug. 29, 2007
(86) PCT No.: PCT/IB2007/004400

§ 371 Date: Oct. 19, 2009

Related U.S. Application Data
(60) Provisional application No. 60/841,138, filed on Aug. 29, 2006.

(51) Int. Cl. G01N 33/53; G01N 33/68

(52) U.S. Cl. 435/7.92; 436/86

(57) ABSTRACT

Disclosed are a method and reagents for diagnosis of anti phospholipid syndrome based on the levels of certain anti 1 can antibodies.

### FIELD OF THE INVENTION

The invention relates generally to a method for diagnosing diseases by detecting levels of antibodies to glycans in a subject. More particularly, the invention relates to methods for diagnosing anti-phospholipid syndrome (APS).

### BACKGROUND OF THE INVENTION

Antiphospholipid syndrome (APS), a disorder characterized by pregnancy morbidity and thrombosis in young individuals, is diagnosed by detection of anti-cardiolipin antibodies or lupus anticoagulant using laboratory tests. Correct identification of patients with this syndrome is important as prophylactic anticoagulant therapy can prevent recurrent, thrombosis and reduce complications during pregnancy.

There are two main classifications of APS. If the patient has an underlying autoimmune disorder, such as systemic lupus erythematosus, the patient is said to have secondary APS. If the patient has no known underlying autoimmune disorder, it is termed primary APS.

APS is characterized by venous or arterial thrombosis—a condition where clots, called thrombi, form in the blood vessels; recurrent miscarriages—the repeated loss of the fetus in pregnancies; and thrombocytopenia—a low number of blood platelets that can lead to bleeding, seen as bruising and tiny red dots on the skin. Patients with APS also may experience symptoms of stroke such as transient ischemic attacks (TIAs). APS patients can be stratified based on their clinical phenotype: Pregnancy loss (PL) for women; Thrombosis (Thr), Central nervous system involvement (CNS).

APS is typically diagnosed based on the clinical manifestations noted above and on laboratory test results. A blood sample is analyzed for the presence of antibodies that react with naturally occurring proteins complexed with phospholipids. These are called antiphospholipid antibodies or anti-cardiolipin antibodies—cardiolipin is one type of phospholipid used in lab tests. Sometimes these antibodies are called lupus anticoagulants when cloning assays are used for their detection. Anti-cardiolipin antibodies from APS patients recognize native beta 2 glycoprotein I (B2GPI), an epitope structurally defined by both cardiolipin and G2GPI, or modified B2GPI and not cardiolipin However, diagnostic methods for APS using B2GPI and Cardiolipin autoantibodies for diagnosing APS show low sensitivity and specificity. For better management of disease there is a clinical need for better diagnosis and prognosis at an earlier stage of the disease.

### SUMMARY OF THE INVENTION

The invention is based in part on the identification of anti glycan antibodies that are specific to APS patients that can be used for diagnosis and/stratification of specific APS phenotypes.

In one aspect, the invention provides a method for diagnosing anti-phospholipid syndrome in a subject. The method includes providing a test sample from a subject and detecting in the test sample an one or more of an anti-β-GlcNAc (GNb) antibody, an anti-β-GalNAc (ANb) antibody, an anti-α-Neu5NAc (NNa) antibody, and an anti-Gal(β1,4)GlcNAc(β) (Ab4GNb) antibody. Levels of the antibody or antibodies are compared to the level of the antibody or antibodies in a control sample obtained from a subject known to not have anti-phospholipid syndrome. Higher levels of the antibody in the test sample as compared to the levels of the antibodies in the control sample indicates the subject has anti-phospholipid syndrome.

In some embodiments, the antibody isotypes include: anti-β-GlcNAc (GNb) IgG antibody, an anti-β-GalNAc (ANb) IgG antibody, an anti-α-Neu5NAc (NNa) IgG antibody, and/or an anti-Gal(β1,4)GlcNAc(β) (Ab4GNb) IgG.

In some embodiments, two, three or four of the an anti-GNb IgG antibody, an anti-ANb IgG antibody, an anti-NNa antibody, and an anti-Ab4GNb IgG antibody are detected.

In some embodiments, the anti-GNb antibody, anti-ANb antibody, anti-NNa antibody, and the anti-Ab4GNb antibody detected are of IgA or IgM type.

In some embodiments, the method includes detecting a native Beta 2-GPI autoantibody in the subject, wherein the presence of the antibody indicates the subject has APS.

In some embodiments, the method includes detecting a cardiolipin autoantibody in the subject, wherein the presence of the antibody indicates the subject has APS.

In some embodiment, the method includes detecting a lupus anti coagulant in the subject, wherein the presence of the antibody indicates the subject has APS.

The test sample can be, e.g., a biological fluid. The biological fluid can be, e.g., whole blood, serum, plasma, urine, or saliva.

In some embodiments, the antibody is detected using a fluorescent antibody.

In some embodiments, the antibody is detected using an enzyme-linked immunoabsorbent assay (ELISA).

Also provided by the invention is a method for prognosing a female with anti-phospholipid syndrome who is at risk for pregnancy loss. The method includes providing a test sample from a pregnant female with anti-phospholipid syndrome and detecting in the test sample an anti-ANb IgG antibody. Levels of the antibody are compared to the level of the antibody in a control sample obtained from pregnant female with anti-phospholipid syndrome who is not at risk for pregnancy loss. Higher levels of the antibody in the test sample as compared to the levels of the antibodies in the control sample indicates the subject is at risk for pregnancy loss. In some embodiments, the female is determined to be at risk for pregnancy loss when the level of an anti-β-GalNAc IgG antibody is above D, wherein D is selected to achieve an optimized clinical parameter selected from the group consisting of: sensitivity, specificity, negative predictive value, positive predictive value and overall agreement. In some embodiments, the pregnancy is a recurrent pregnancy.

Also provided by the invention is a method for identifying a patient with anti-phospholipid syndrome who is at risk for thrombosis. The method includes providing a test sample from a patient with anti-phospholipid syndrome and detecting in the test sample one or more of an anti-ANb antibody, anti GNb, anti NNa, and anti-Ab4GNb. The amount of antibodies are compared to the level of the antibodies in a control sample obtained from patient with anti-phospholipid syndrome who is at risk for thrombosis. Similar level of the antibodies in the test sample as compared to the levels of the antibodies in the control sample indicates the subject is at risk for thrombosis.

Also provided by the invention is a method for identifying patients with anti-phospholipid syndrome who is at risk for CNS involvement. The method includes providing a test sample from a patient with anti-phospholipid syndrome and detecting in the test sample an one or more of an anti-ANb, anti-GNb, anti-NNa, or anti-Ab4GNb levels. The amounts antibodies are compared to the amounts of the antibodies in a control sample obtained from patient with anti-phospholipid syndrome who is at risk for CNS involvement. Similar level of the antibodies in the test sample as compared to the levels of the antibodies in the control sample indicates the subject is at risk for CNS involvement.

The test sample can be, e.g., a biological fluid. The biological fluid can be, e.g., whole blood, serum, plasma, urine, or saliva.

In some embodiments, the antibody is detected using a fluorescent antibody.

In some embodiments, the antibody is detected using an enzyme-linked immunoabsorbent assay (ELISA).

Also provided by the invention is software stored in a computer storage medium for diagnosing anti-phospholipid syndrome in a subject. The software is operable to receive for a subject with symptoms of APS data for levels in a sample from the subject of one or more of an anti-GNb IgG antibody, an anti-ANb IgG antibody, an anti-ANa IgG antibody, an anti-NNa antibody, and an anti-Ab4GNb IgG antibody. The software compares levels of the antibody to levels of the antibody to the level of the antibody in a control sample obtained from a subject known to not have anti-phospholipid and determines that the subject has anti-phospholipid syndrome if higher levels of the antibody are detected in the test sample as compared to the levels of the antibodies in the control sample.

Also provided by the invention is a system for diagnosing anti-phospholipid syndrome in a subject. The system includes at least one memory operable to store data for levels in a sample from the subject of one or more of an anti-GNb IgG antibody, an anti-ANb IgG antibody, an anti-ANa IgG antibody, an anti-NNa antibody, and an anti-Ab4GNb IgG antibody. The system also includes one or more processors, collectively operable to compare levels of the antibody to levels of the antibody to the level of the antibody in a control sample obtained from a subject known to not have anti-phospholipid syndrome and to determine that the subject has anti-phospholipid syndrome if higher levels of the antibody are detected in the test sample as compared to the levels of the antibodies in the control sample.

Also within the invention are substrates that include reagents that specifically detect the antibodies disclosed herein, e.g., an anti-β-GalNAc antibody, an anti-α-Neu5NAc antibody, and/or an anti-Gal(β1,4)GlcNAc(β). In some embodiments, the substrates additionally include reagents that detect a β-GlcNAc antibody, a native Beta 2-GPI.autoantibody, a cardiolipin antibody, and/or a lupus anti coagulant.

Also within the invention is substrate that includes a reagent that can specifically detect a (β-GalNAc antibody.

The substrate can be, e.g., planar. In a further aspect, the reagents may be connected to a substrate via a linker.

In a further aspect, the reagents may be connected to a substrate via a linker. The substrate may be a bead particles or a planer substrate.

The invention additionally provides a kit that include reagents for detecting anti-glycan antibodies that reveal the presence of APS. The kit includes one or more carbohydrate reagent(s) that specifically reacts with an anti-β-GalNAc antibody, an anti-α-Neu5NAc antibody, and/or an anti-Gal(β1,4)GlcNAc(β) antibody. The kits may be provided in one or more containers. In some embodiments, the kits contain directions for using the kits to perform the methods described herein. The kits may optionally include reagents for detecting antibody isotypes (e.g., IgA, IgG, and IgM antibodies).

In some embodiments, the kits include reagents that are used to specifically bind and detect those anti glycans antibodies that are the specific glycan structures. In other embodiments, the reagents in the kits are other molecules or macromolecules that include the specific glycan structure. For example, the anti-β-GalNAc antibody can be detected using the polysaccharide of the cell wall of Viridans streptococci. Thus, the glycan itself can be used for detecting the corresponding antibody or antibodies, as can any carbohydrate, peptide, protein, or any other molecular structure that includes the glycan.

The kits may optionally also include reagents that specifically detect an β-GlcNAc antibody, a native Beta 2-GPI.autoantibody, a cardiolipin antibody, and/or a lupus anti coagulant.

Also provided by the invention is a kit for prognosing a female with anti-phospholipid syndrome who is at risk for pregnancy loss. The kit includes a reagent that detects an anti-β-GalNAc antibody and, optionally, directions for using the kit.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a graph showing levels of anti GNb, ANb, ANa, NNa, and Ab4GNb IgG in APS patients versus individuals without APS. The mean, median, standard deviation anti glycan O.D. levels, and the p value vs APS groups are shown in the table iii lower part of the figure.

FIG. 2 is a graph showing anti ANb IgG levels in a group of women with pregnancy loss (PL) and in a group without PL. The mean, median, standard deviation anti glycan O.D. levels, and the p value vs PL group (designated as group 1) are shown in the table in lower part of the figure.

FIG. 3 is a ROC curve analysis using ANb levels to differentiate between APS females that experience pregnancy loss and those who do not experience pregnancy loss.

FIG. 4 is a graph showing anti ANb levels in APS, SLE and normal groups.

FIG. 5 is a ROC curve analysis using ANb IgG levels to differentiate between APS and control (SLE+normal) groups.

FIG. 6 is a graph showing the correlation between anti ANa and anti ANb IgG in APS and control population groups.

FIG. 7 is a graph showing a lack of correlation between anti ANb O.D. and anti Beta 2 GPI units in APS patients.

### DETAILED DESCRIPTION OF THE INVENTION

The invention provides methods for diagnosing and stratifying anti-phospholipid syndrome (APS) by examining a test sample from a subject for antibodies to one or more, specific glycans, and diagnosing APS based on the level of the antibodies in the patient.

Certain antibodies to glycan structures are discussed herein. The glycans are presented either in the International Union of Pure and Applied Chemistry (IUPAC) condensed form for nomenclature carbohydrate representation or in LINEARCODE® syntax, for linear code syntax principles see (Banin et al., Trends in Glycoscience and Glycotechnology, 14:127-37, 2002). A translation of the LINEARCODE® representation to IUPAC representation is presented in Table 1. All the glycan structures that discussed herein, unless mentioned otherwise, are connected in the indicated anomericity α or β to another molecular structure, linker, or solid phase.

In some embodiments, the reagents that are used to specifically bind and detect those anti glycans antibodies are the specific glycan structures. In other embodiments, the reagents are other molecules or macromolecules that include the specific glycan structure. The glycan or sugar structures can be only the a carbohydrate moiety (including monosaccharides an oligosaccharide or a polysaccharide) or displaying on any solid phase or other macromoleculeor any other molecular structure that includes the glycan. The glycan-containing structure can be obtained from natural sources, e.g., extracted from an organism, or can be prepared syntheticaly.

For example, an anti-Glc(β1,3)Glc(β) antibody can be detected using the polysaccharide β-D(1,3) Glucan, a polymer of glucose units connected in a (β1,3) glycosidic bond. Thus, the glycan itself can be used for detecting the corresponding antibody or antibodies, as can any carbohydrate, peptide, protein, or any other molecular structure that includes the glycan.

In some embodiments, the reagents that are used to specifically bind and detect the anti glycans antibodies of the invention are peptides that mimic the carbohydrate antigens of the invention. The peptides can be used to identify specific anti glycan antibodies.

### Generating an Anti-Glycan Antibody Profile

An anti-glycan antibody profile is generated using a sample obtained from the subject to be diagnosed. The term “anti-glycan antibody profile,” (AGAP) as used herein, means the levels of one or more anti glycan antibodies in a sample. The term “sample,” as used herein, means any biological specimen obtained from an individual that contains antibodies. A sample can be, for example, whole blood, plasma, saliva or other bodily fluid or tissue having antibodies, preferably a serum sample. Samples can be diluted if desired before they are analyzed for anti-glycan antibodies. The subject can be, e.g., a human, a non-human primate (including a chimpanzee, ape, gorilla, old world primate), cow, horse, dog, cat, pig, goat, sheep, rodent (including, e.g., a mouse, rat, or guinea pig) Anti-glycan profiles can be determined by using methods known in the art for identifying antibodies to glycans. The methods include those disclosed in e.g., U.S. Pat. No. 6,972,172, or Schwarz et al., Glycobiology 13:749-54, 2003, or Dotan et al. Lupus 15:443-50, 2006.

The methods are typically performed using reagents that specifically bind to the anti-glycan antibodies. The reagents can be, e.g., the specific glycan structures. Alternatively, the reagents can be other molecules or macromolecules that include the specific glycan structure. For example, the anti-Glc(β1,3)Glc(β) antibody can be detected using the polysaccharide β-D(1,3)Glucan, a polymer of glucose units connected in a (β1,3)glycosidic bond. Thus, the glycan itself can be used for detecting the corresponding antibody or antibodies, as can any carbohydrate, peptide, protein, or any other molecular structure that includes the glycan.

If desired, the peptides that mimic carbohydrate antigens can be used in the methods and compositions described herein. The peptides can be used to identify specific anti glycan antibodies. Peptides which mimic structures recognized by antiglycan antibodies can be identified using methods known in the art, e.g., by screening a filamentous phage-displayed random peptide library (Zhan et al., Biochem Biophys Res Commun. 308:19-22, 2003; Hou et al., J. Immunol. 17:4373-79, 2003).

Glycan antigens used to identify various anti-glycan antibodies can be obtained from a variety of other sources so long as the antigen is capable of binding specifically to the given anti-glycan antibody. Binding to anti-glycan antibodies can be performed using variety of other immunoassay formats known in the art, including competitive and non-competitive immunoassay formats can also be used (Self and Cook, Curr. Opin. Biotechnol. 7:60-65 (1996), which is incorporated by reference). Other assays include immunoassays, such as enzyme-linked immunosorbent assays (ELISAs). An enzyme such as horseradish peroxidase (HRP), alkaline phosphatase (AP), β-galactosidase or urease can be linked to a secondary antibody selective for a primary anti-glycan antibody of interest. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-a β-D-galactopyranoside (ONPG), which yields a soluble product detectable at 410 nm, or a urease detection system can be used with a substrate such as urea-bromocresol purple (Sigma Immunochemicals, St. Louis, Mo.). A useful secondary antibody linked to an enzyme can be obtained from a number of commercial sources; goat F(ab′)₂ anti-human IgG-alkaline phosphatase, for example, can be purchased from Jackson Immuno-Research (West Grove, Pa.).

Immunoassays encompass capillary electrophoresis based immunoassays (CEIA) and can be automated, if desired. Immunoassays also can be used in conjunction with laser induced fluorescence (see, for example, Schrnalzing and Nashabeh, Electrophoresis 18:2184-93 (1997)); Bao, J. Chromatogr. B. Biomed. Sci. 699:463-80 (1997), each of which is incorporated herein by reference). Liposome immunoassays, such as flow-injection liposome immunoassays and liposome immunosensors, also can be used (Rongen et al., J. Immunol. Methods 204:105-133 (1997)).

A radioimmunoassay can also be used for determining whether a sample is positive for a glycan antibody, or for determining the level of anti-glycan antibodies in a sample. A radioimmunoassay using, for example, an ¹²⁵Iodine-labeled secondary antibody (Harlow and Lane, Antibodies A Laboratory Manual Cold Spring Harbor Laboratory: New York, 1988, which is incorporated herein by reference) is encompassed within the invention.

A secondary antibody may alternatively be labeled with a chemiluminescent marker. Such a chemiluminescent secondary antibody is convenient for sensitive, non-radioactive detection of anti-glycan antibodies and can be obtained commercially from various sources such as Amersham Lifesciences, Inc. (Arlington Heights, Ill.).

A detectable reagent may also be labeled with a fluorochrome. Appropriate fluorochromes include, for example, DAPI, fluorescein, Hoechst. 33258, R-phycocyanin, B-phycoerythrin, R-phycoerythrin, rhodamine, Texas red or lissamine. A particularly useful fluorochrome is fluorescein or rhodamine. Secondary antibodies linked to fluorochromes can be obtained commercially. For example, goat F(ab′)₂ anti-human IgG-FITC is available from Tago Immunologicals (Burlingame, Calif.).

A signal from the detectable reagent can be analyzed, for example, using a spectrophotometer to detect color from a chromogenic substrate; a radiation counter to detect radiation, such as a gamma counter for detection of ¹²⁵Iodine; or a fluorometer to detect fluorescence in the presence of light of a certain wavelength. For detection of enzyme-linked reagents, a quantitative analysis of the amount of anti-glycan antibodies can be made using a spectrophotometer such as an EMAX Microplate Reader (Molecular Devices, Menlo Park, Calif.) in accordance with the manufacturer's instructions. If desired, the assays of the invention can be automated or performed robotically, and the signal from multiple samples can be detected simultaneously.

Other methods include, e.g., flow cytometry (including bead based immunoassays), and phage display technology for expressing a recombinant antigen specific for an anti-glycan antibody. Phage particles expressing the antigen specific for a desired anti-glycan antibody can be anchored, if desired, to a multiwell plate using an antibody such as an anti phage monoclonal antibody (Felici et al., “Phage-Displayed Peptides as Tools for Characterization of Human Sera” in Abelson (Ed.), Methods in Enzymol. 267, San Diego: Academic Press, Inc. (1996), which is incorporated by reference herein).

Anti-glycan antibodies are conveniently detected by simultaneously analyzing multiple sample for the presence of one or more anti-glycan antibodies. For example, the antibodies can be detected using an array of reagents that can bind specifically to the anti glycan antibodies. Preferably, each reagent is provided in a different location with a defined address on the array. By exposing the sample to array all the anti glycan antibodies that bind to the reagent on the array can be detected in one test Suitable arrays that include reagents (preferably carbohydrate reagents) that specifically detect the APS-detecting antibodies disclosed herein, e.g., an anti-β-GalNAc IgG antibody for diagnosing APS.

In some embodiments, the reagents that are used to specifically bind and detect those anti glycans antibodies are displayed on tagged beads, enabling to test in one experiment the levels of various anti glycan antibodies. For example, tagged beads multiplexed assay systems are described in Kellar et al Ex.p Hematol. 30:1227-37, 2002.

In some embodiments, the reagents that are used to specifically bind and detect those anti glycans antibodies are the specific glycan structures. In other embodiments, the reagents are other molecules or macromolecules that include the specific glycan structure. For example, the kits are other molecules or macromolecules that include the specific glycan structure. For example, the anti-β-GalNAc antibody can be detected using the polysaccharide of the cell wall of Viridans streptococci, which contains β-GalNAc (Cisar et al. Glycobiology, 1995 October; 5(7):655-62). Thus, the glycan itself can be used for detecting the corresponding antibody or antibodies, as can any carbohydrate, peptide, protein, or any other molecular structure that includes the glycan.

In some embodiments, the glycans are attached to the array via a linker. A suitable linker includes at least one ethylene glycol derivative, at least two cyanuric chloride derivatives and an anilino group.

If desired, peptides that mimic carbohydrate antigens can be used in the methods and compositions described herein. The peptides can be used to identify specific anti glycan antibodies. Peptides which mimic structures recognized by antiglycan antibodies can be identified using methods known in the art, e.g., by screening a filamentous phage-displayed random peptide library (Than et al., Biochem Biophys Res Commun. 308:19-22, 2003; Hou et al., J Immunol. 17:4373-79, 2003.)

### Interpreting Anti-Glycan Antibody Binding Data

Typically, binding of anti-glycan antibodies to glycans in a sample is compared to a reference population, and differences in levels of the anti-glycan antibodies in the two samples are compared. The threshold for determining whether a test sample is scored positive for APS or non-APS can be altered depending on the sensitivity or specificity desired. The clinical parameters of sensitivity, specificity, negative predictive value, positive predictive value and overall agreement are calculated using true positives, false positives, false negatives and true negatives. A “true positive” sample is a sample positive for APS according to the presence of clinical symptoms, and/or sera analysis for the presence of anti cardiolipin antibodies or lupus anticoagulant, which is also diagnosed positive according to a method of the invention. A “false positive” sample is a sample negative for APS by presence of clinical symptoms, and/or sera analysis for the presence of anti cardiolipin antibodies or lupus anticoagulant, which is diagnosed positive according to a method of the invention. Similarly, a “false negative” is a sample positive for APS by presence of clinical symptoms, and/or sera analysis for the presence of anti cardiolipin antibodies or lupus anticoagulant, which is diagnosed negative according to a method of the invention. A “true negative” is a sample negative for APS by presence of clinical symptoms, and/or sera analysis for the presence of anti cardiolipin antibodies or lupus anticoagulant, and also negative for APS according to a method of the invention. See, for example, Mousy (Ed.), Intuitive Biostatistics New York: Oxford University Press (1995), which is incorporated herein by reference.

As used herein, the term “sensitivity” means the probability that a laboratory method is positive in the presence of APS. Sensitivity is calculated as the number of true positive results divided by the sum of the true positives and false negatives. Sensitivity essentially is a measure of how well a method correctly identifies those with disease. In a method of the invention, the anti-glycan antibody values can be selected such that the sensitivity of diagnosing an individual is at least about 60%, and can be, for example, at least about 65%, 70%, 75%, 80%, 85%, 90% or 95%.

As used herein, the term “specificity” means the probability that a method is negative in the absence of APS. Specificity is calculated as the number of true negative results divided by the sum of the true negatives and false positives. Specificity essentially is a measure of how well a method excludes those who do not have APS. The anti-glycan cut-off value can be selected such that, when the sensitivity is at least about 70%, the specificity of diagnosing an individual is in the range of 30-60%, for example, 35-60%, 40-60%, 45-60% or 50-60%.

The term “positive predictive value,” as used herein, is synonymous with “PPV” and means the probability that an individual diagnosed as having APS actually has the disease. Positive predictive value can be calculated as the number of true positives divided by the sum of the true positives and false positives. Positive predictive value is determined by the characteristics of the diagnostic method as well as the prevalence of the disease in the population analyzed. In a method of the invention, the anti-glycan antibody cut-off values can be selected such that the positive predictive value of the method in a population having a APS disease prevalence of 15% is at least about 5%, and can be, for example, at least about 8%, 10%, 15%, 20%, 25%, 30% or 40%.

As used herein, the term “efficiency” means the accuracy with which a method diagnoses a disease state. Efficiency is calculated as the sum of the true positives and true negatives divided by the total number of sample results and is affected by the prevalence of APS in the population analyzed. The anti-glycan antibody cut-off values can be selected such that the overall agreement of a method of the invention in a patient population having an APS disease prevalence of 15% is at least about 45%, and can be, for example, at least about 50%, 55% or 60%.

In some embodiments, a subject is determined to have APS if the level of the measured antibody or antibodies is above a cut-off value, which can be independently determined for each antibody. The cut-off values can be independently selected to achieve an optimized clinical parameter including, e.g., sensitivity, specificity, negative predictive value, positive predictive value and overall agreement. For example, when a sample is contacted with antibodies to two or more of an anti-GNb antibody, an anti-ANb antibody, an anti-NNa antibody, and/or an anti-Ab4GNb antibody, a diagnosis of APS can be made if the level of ANb antibody is above A, the level of an anti-ANb antibody is above B, the level of an anti-NNa anti-body is above C, and/or the level of an anti-Ab4GNb antibody is above D, wherein A, B, C, and D are independently selected to achieve an optimized clinical parameter selected from the group consisting of: sensitivity, specificity, negative predictive value, positive predictive value and overall agreement.

The invention will be further illustrated in the following non-limiting examples.

### Example 1

### Diagnosing and Staging APS Using Anti-glycan Antibodies

Frozen sera from clinically characterized primary APS patients (n=116), systemic lupus erythematosus (SLE) patients (n=103) not having secondary APS, and a healthy control group (n=72) were screened for the presence of a set of anti glycan IgG antibodies using an enzyme immune assay (see the list of glycans in Table 1 and the demographic characteristics of patients in Table 2). The screening was done using ELISA based assays as follows:

Glycans p-nitrophenyl derivatives were covalently attached to the surface of a clear 96-well microtiter plate as previously described (Schwarz et al., Glycobiology 13:749-54, 2003). Serum samples were diluted 1:100 in a buffer (SDB cat. G300023, Glycominds, Lod, Israel), dispensed into the wells (50 μL per well) incubated for 30 min at 25° C., then washed with PBST buffer. Bound antibodies were labeled (30 min at 25° C.) with 50 μL of either horseradish peroxidase (HRP)-conjugated goat anti-human IgG (1:25000) type-specific antibody (Jackson, ImmunoResearch Laboratories, West Grove, Pa., USA), washed with PBST buffer. 50 μL 3,3′,5,5′-tetramethylbenzidine (TMB) was added for detection. The optical density (OD) at 595 nm was read after 15 min with a Victor 1420 plate reader (Wallac, Turku, Finland). The enzymatic reaction was stopped with 50 μM sulfuric acid solution and read at 450 nm. T-test was used to calculate significant difference between groups.

APS Vs. Normal Patients

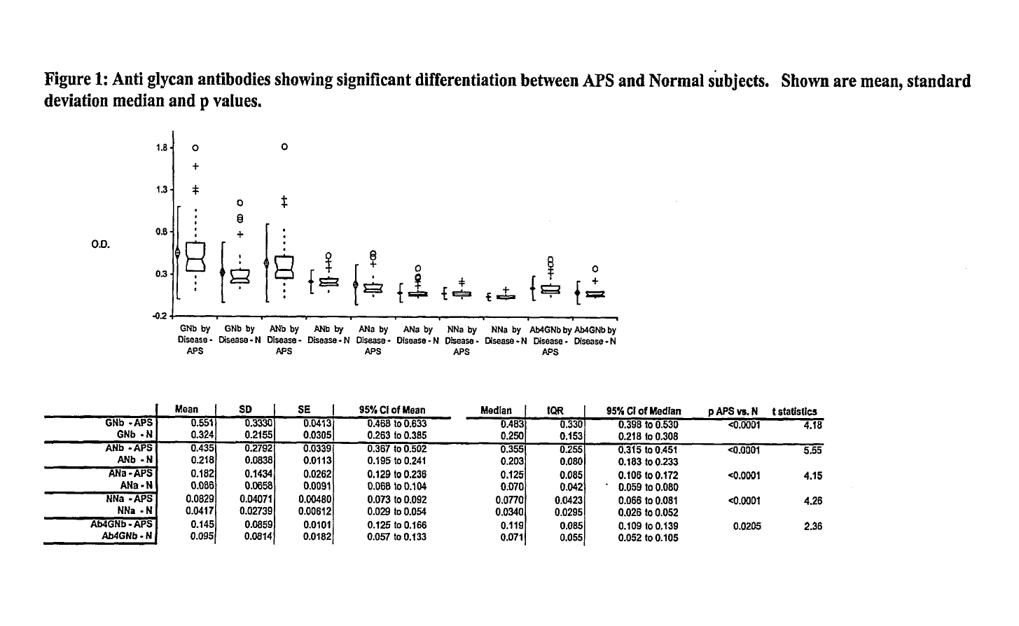

A significantly higher level of anti GNb, ANb, ANa, NNa, and Ab4GNb IgG (p<0.05) were found in APS patients as compared to normal patients (FIG. 1).

### Stratification of APS Patients—Pregnancy Loss

The cohort included 45 APS females that did not experience pregnancy loss (PL) and 28 who did. The levels of all anti glycan antibodies were compared between the females groups (PL and no PL).

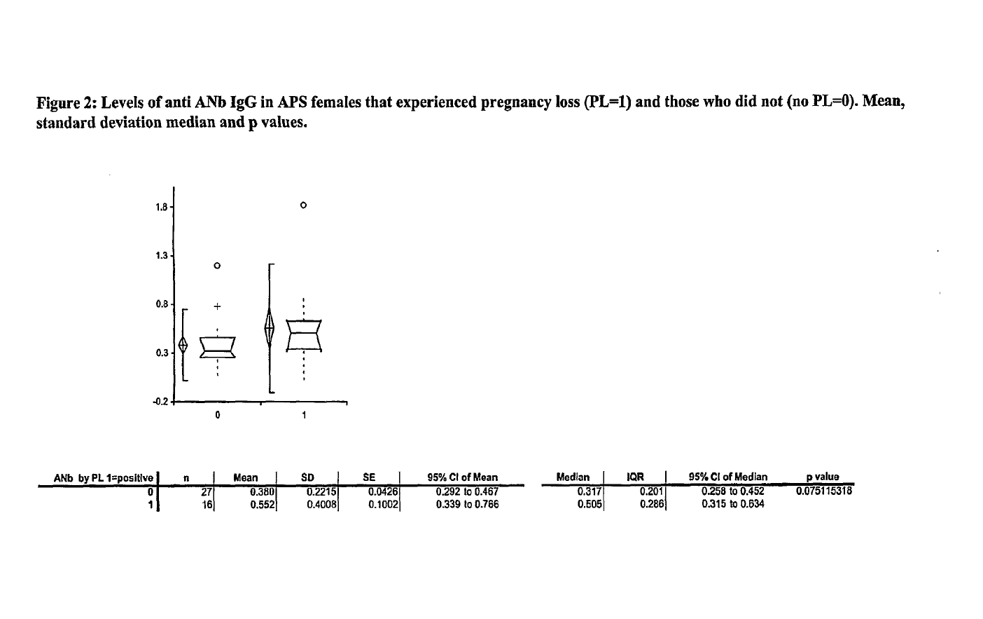

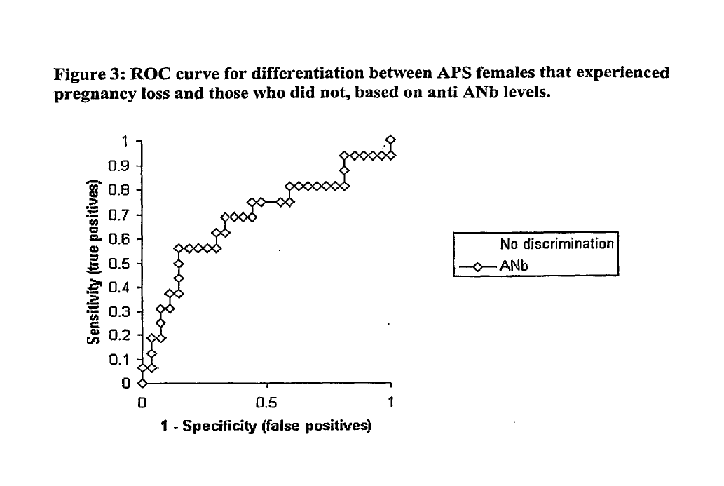

Anti ANb IgG levels in the PL group were higher than in the non PL group (almost reaching significance, p=0.07), see FIG. 2. However, ROC curves analysis for differentiation between the groups shows the difference between Anti ANb IgG levels enable significant separation between PL and non PL in sensitivity (56%) and specificity (85%) AUC=0.68, p=0.02. See FIG. 3.

### APS Vs SLE and Normal Controls

To further validate these findings we the IgG anti ANb and anti ANa levels in APS, SLE, and healthy normal controls were screened.

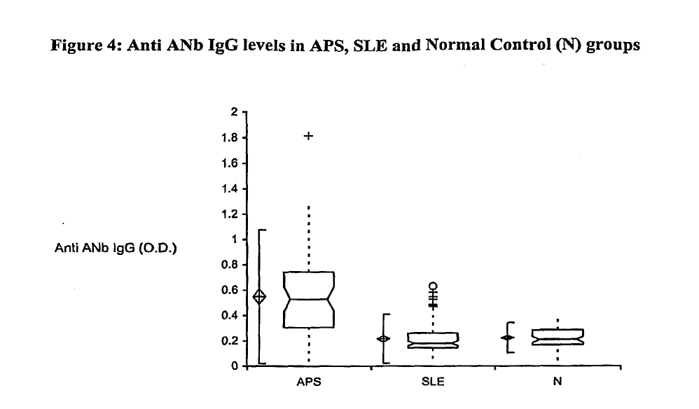

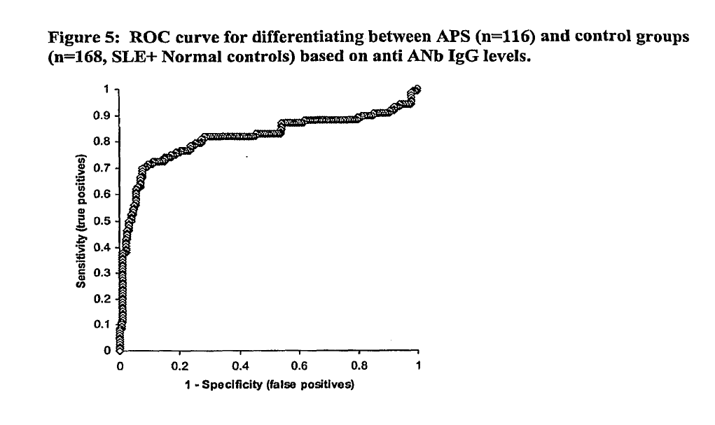

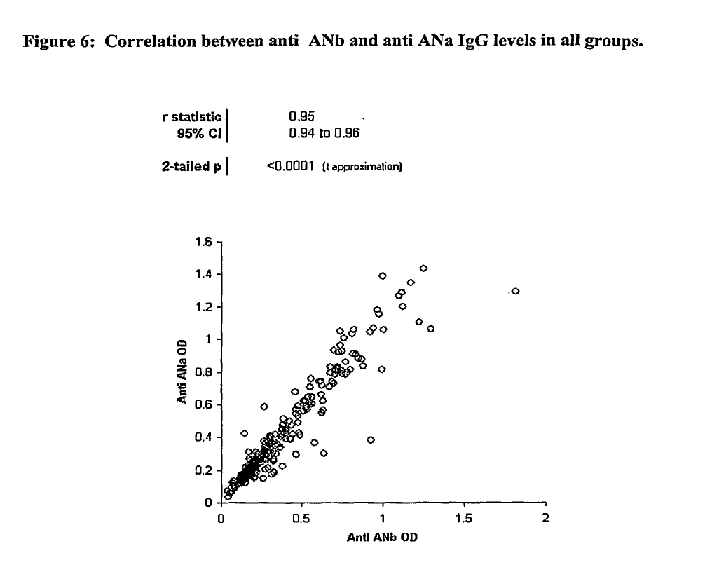

Significantly higher levels (p<0.0001) of anti ANb IgG levels were found in the APS group in comparison to the SLE and control group. Mean ANb levels in the different groups are described in Table 3 and FIG. 4. ROC curve analysis describing the differentiation between APS and control group is described in FIG. 5. Cutoff levels of 0.33 O.D. enables differentiation between groups in sensitivity of 72%, specificity 90%, positive predictive value 84%, and negative predictive value of 83%. The correlation between anti ANa and anti ANb in this comparison was very high (FIG. 6), demonstrating that both alpha and beta anomers of GalNAc can be used to identify the antibodies.

Levels of anti GNb, ANb, ANa, NNa, and Ab4GNb IgG antibodies yielded a very significant difference between APS and normal groups. ANb further differentiated between females who had PL and those who did not. Anti ANb and ANa were significantly higher in APS group in comparison to a group of SLE patients enabling differentiation between APS and controls (SLE plus normal healthy controls) in high sensitivity and specificity.

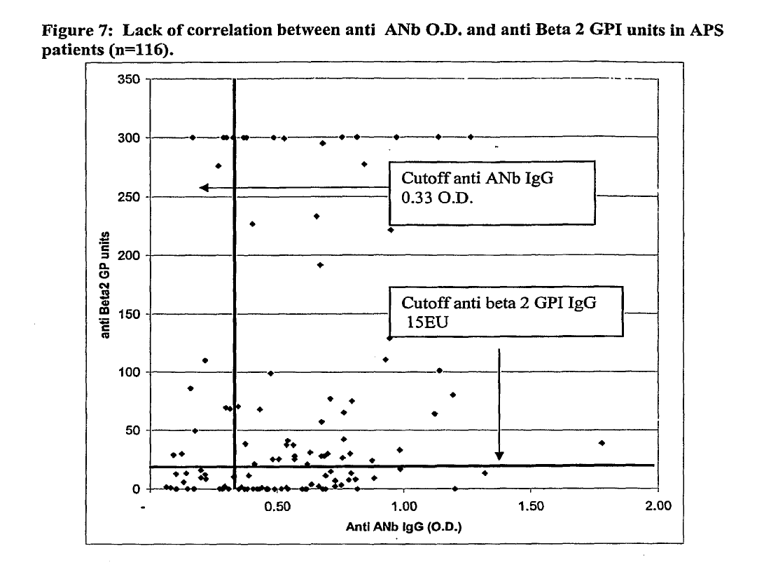

Lack of correlation between levels of anti Beta 2GPI IgG and anti ANb IgG in APS patients. We measured the levels of anti beta 2GPI IgG in the APS group using commercial ELISA kits for measuring anti beta 2GPI IgG, and compared the levels of anti ANb IgG levels. As can be seen in FIG. 7, the correlation between anti ANb IgG and anti beta 2GPI IgG is low. Furthermore, when using cutoff levels of 15EU for anti beta 2GPI IgG (according to the manufacturer's manual), and 0.33 O.D. for anti ANb, 65% (35/54) of the APS patients that are anti beta 2GPI IgG negative were positive for anti ANb (FIG. 7). When combing anti ANb and anti beta 2GPI IgG 83% of the APS population are positive in one of the assays.

Human beta 2GPI is a heavily glycosylated five-domain plasma membrane-adhesion protein. However the glycans decoration of beta 2GPI does not contains any GalNAc (Ph.D. thesis of Bouma, Barend “Structural studies on b2-glycoprotein I and von Willebrand factor A3 domain” University of Utrecht 2000 ISBN 90.393.2472.7). It was surprising and not predicted to find anti GalNAc antibodies in APS patients. The lack of correlation between anti beta 2GPI support the idea that the anti GalNAc IgG were induced due to other antigen then beta 2GPI.

TABLE 1
Glycans examined

| Glycan | |
|---|---|
| abbreviations | |
| using | |
| LINEARCODE ® | Full name |
| GNb | β-GlcNAc |
| GNbGNb | GlcNAc(β1,4) GlcNAc(β) |
| Ab | β-Gal |
| ANb | β-GalNAc |
| Ana | α-GalNAc |
| Gb3Gb | Glc(β1,3)Glc(β) |
| Ab4GNb | Gal(β1,4)GlcNAc(β) |
| Ab4Gb | Gal(β1,4)Glc(β) |
| GNa | α-GlcNAc |
| Ab3Ana | Gal(β1,3)GalNAc(α) |
| Ma6Ma | Man(α1,6)Man(α) |
| NNa | α-Neu5NAc |

TABLE 2
Patient characteristics.

| | APS | SLE | HC |
|---|---|---|---|
| | (n = 116) | (n = 96) | (n = 72) |
| Mean age, years (SD) | 42.4 (11.9) | 47.2 (14.2) | 43.7 (11.5) |
| Female, n (%) | 84 (73) | 80 (83) | 50 (70) |

SLE group does not include patients with SLE and APS.

TABLE 3
Anti ANb IgG in APS Patients and controls

| | APS | SLE | HC |
|---|---|---|---|
| | (n = 116) | (n = 96) | (n = 72) |
| IgG Anti ANb levels, O.D. (SD) | 035 (0.32) | 0.21 (0.11)* | 0.22(0.07)* |

*p <0.0001 versus APS. SLE group does not include patients with SLE and APS.

The descriptions given are intended to exemplify, but not limit, the scope of the invention. Additional embodiments are within the claims.

1. A method for diagnosing anti-phospholipid syndrome (APS) in a subject, the method comprising, providing a test sample from a subject, detecting in said test sample at least one antibody selected from the group consisting of an anti-β-GalNAc antibody, an anti-α-Neu5NAc antibody, and an anti-Gal(β1,4)GlcNAc(β) antibody; and comparing the levels of said antibody to the level of said antibody in a control sample obtained from a subject known to not have anti-phospholipid syndrome, wherein higher levels of said antibody in said test sample as compared to the levels of said antibodies in said control sample from a subject not having anti-phospholipid syndrome indicates said subject has anti-phospholipid syndrome.

2. The method of claim 1, wherein the subject is determined to have APS when the level of anti-β-GalNAc antibody is above A, the level of an anti-α-Neu5NAc antibody is above B, the level of an anti-Gal(β1,4)GlcNAc(β) antibody is above C, wherein A, B, and C, are independently selected to achieve an optimized clinical parameter selected from the group consisting of: sensitivity, specificity, negative predictive value, positive predictive value and overall agreement.

3. The method of claim 1, wherein said antibody is an anti-β-GalNAc antibody.

4. The method of claim 3, wherein the subject is determined to have APS when the level of anti-β-GalNAc antibody is above A wherein A is selected to achieve an optimized clinical parameter selected from the group consisting of: sensitivity, specificity, negative predictive value, positive predictive value and overall agreement.

5. The method of claim 1, wherein said method comprises detecting two of said antibodies and comparing the levels of said antibodies to the levels of said antibodies in said control sample, and wherein higher levels of said antibodies in said test sample as compared to the levels of said antibodies in said control sample indicates said subject has anti-phospholipid syndrome.

6. The method of claim 1, wherein said method comprises detecting three of said antibodies and comparing the levels of said antibodies to the levels of said antibodies in said control sample, and wherein higher levels of said antibodies in said test sample as compared to the levels of said antibodies in said control sample indicates said subject has anti-phospholipid syndrome.

7. The method of claim 1, further comprising detecting a native Beta 2-GPI autoantibody in said subject, wherein the presence of said antibody indicates said subject has APS.

8. The method of claim 1, further comprising detecting a cardiolipin autoantibody in said subject, wherein the presence of said antibody indicates said subject has APS.

9. The method of claim 8, further comprising detecting a native Beta 2-GPI autoantibody in said subject, wherein the presence of said antibody indicates said subject has APS.

10. The method of claim 1, further comprising detecting a lupus anti coagulant in said subject, wherein the presence of said antibody indicates said subject has APS.

11.-14. (canceled)

15. The method of claim 1, wherein said antibody is detected using an enzyme-linked immunoabsorbent assay (ELISA).

16.-19. (canceled)

20. A method for prognosing a female with anti-phospholipid syndrome who is at risk for pregnancy loss, the method comprising, providing a test sample from a pregnant female with anti-phospholipid syndrome, detecting in said test sample an anti-β-GalNAc IgG antibody; and comparing the levels of said antibody to the level of said antibody in a control sample obtained from pregnant female with anti-phospholipid syndrome who is not at risk for pregnancy loss, herein higher levels of said antibody in said test sample as compared to the levels of said antibodies in said control sample indicates said subject is at risk for pregnancy loss.

21. The method of claim 20, wherein the female is determined to be at risk for pregnancy loss when the level of an anti-β-GalNAc IgG antibody is above D, wherein D is selected to achieve an optimized clinical parameter selected from the group consisting of: sensitivity, specificity, negative predictive value, positive predictive value and overall agreement.

22. (canceled)

23. A kit for diagnosing anti-phospholipid syndrome (APS) in subject, the kit comprising: a first reagent that specifically detects one or more of an anti-β-GalNAc antibody, an anti-α-Neu5NAc antibody, and an anti-Gal(β1,4)GlcNAc(β) antibody, and, reagents for determining the isotype of an antibody, and optionally, directions for using said kit.

24. (canceled)

25. The kit of claim 23, further comprising reagents that specifically detect a native Beta 2-GPI. autoantibody, a cardiolipin antibody, and/or a lupus anti coagulant.

26.-27. (canceled)
